(19) United States
(12) Patent Application Publication
(10) Pub. No.: US 2025/0295567 A1
(43) Pub. Date: Sep. 25, 2025

(54) SOLID COSMETIC COMPOSITION

(71) Applicant: COSMETIC WARRIORS LIMITED, Poole Dorset (GB)

(72) Inventors: Alessandro COMMISSO, Poole Dorset (GB); Helen Elizabeth AMBROSEN, Poole Dorset (GB); Mark CONSTANTINE, Poole Dorset (GB); Margaret Joan CONSTANTINE, Poole Dorset (GB)

(73) Assignee: COSMETIC WARRIORS LIMITED, Poole Dorset (GB)

(21) Appl. No.: 18/863,295

(22) PCT Filed: May 5, 2023
(86) PCT No.: PCT/GB2023/051190

§ 371 Date: Nov. 5, 2024

(30) Foreign Application Priority Data
May 6, 2022 (GB) 2206641.9

(51) Int. Cl. A61K 8/37; A61K 8/02; A61K 8/25; A61K 8/26; A61K 8/27; A61K 8/29; A61K 8/39; A61K 8/92; A61Q 19/10

(52) U.S. Cl. CPC A61K 8/375; A61K 8/0216; A61K 8/25; A61K 8/26; A61K 8/27; A61K 8/29; A61K 8/39; A61K 8/922; A61Q 19/10; A61K 2800/30; A61K 2800/43; A61K 2800/805

(57) ABSTRACT

A solid cosmetic composition includes (a) vegetable butter; (b) a first emulsifier having a hydrophilic-lipophilic balance (HLB) value of from 2 to 9; (c) a second emulsifier having an HLB value of from 10 to 20; (d) one or more inert powders in an amount of from 0.5 to 25 wt. % based on the solid cosmetic composition; (e) one or more colors; and (f) one or more fragrances.

### CROSS-REFERENCE TO RELATED APPLICATIONS

The present application is a National Stage Application of PCT International Application No.: PCT/GB2023/051190 filed on May 5, 2023, which claims priority to GB Patent Application 2206641.9, filed in Great Britain on May 6, 2022, the disclosure of which are incorporated herein by reference in their entirety.

### FIELD OF THE INVENTION

The present invention relates to a solid cosmetic product, a process for producing said product, and a product prepared by the method.

### BACKGROUND TO THE INVENTION

The present invention relates to products particularly those for use in contact with the human body.

Therapeutic bathing products are known in the cosmetic industry and are available in a variety of different forms. Bath salts, oils, and lotions are a few examples of bathing products that can deliver emollients, essential oils and fragrances to a bath, providing calming and remedial properties. However, the required use of solid packaging for these products is a significant disadvantage due to the environmental impact that waste packaging has, despite the availability of recycling.

For at least this reason encapsulated compositions for creating therapeutic bath products have become increasingly popular. For example, U.S. Pat. No. 4,294,855 and WO03/061633 disclose compositions which are capable of enveloping a core cosmetic. The active therapeutic components are often encased in a gelatinous or membrane-bound shell, which dissolves on contact with water, releasing the core ingredients into the water. However, the disadvantage of these products is that the shell does not readily dissipate in water, delaying the release of the active ingredients, whilst providing no effective benefit of its own. They are also prone to rupturing, resulting in a wasted and suboptimal product.

In addition to the problems of providing bathing products which do not require unnecessary packaging, it is also found that users sometimes wish to enjoy the fragrance of their purchased product without the need to bathe in the product. To achieve this aim, it is often necessary for users to buy a separate fragranced product such as a “reed diffuser”. This results in further cost to the user and a potential for further unnecessary packaging and transport waste.

The present invention seeks to provide a solid cosmetic product that may be used as a therapeutic bathing product and is capable of delivering a concentrated dose of emollients, essential oils and/or fragrances with an enhanced experience for the user, and which also may be used to deliver fragrance other than when the user is bathing.

### SUMMARY OF THE INVENTION

In one aspect there is provided a solid cosmetic composition comprising
(a) vegetable butter;
(b) a first emulsifier having a HLB value of from 2 to 9;
(c) a second emulsifier having a HLB value of from 10 to 20;
(d) one or more inert powders in an amount of from 0.5 to 25 wt. % based on the solid cosmetic composition;
(e) one or more colours; and
(f) one or more fragrances.

In one aspect there is provided a solid cosmetic composition comprising
(a) vegetable butter;
(b) a first emulsifier having a HLB value of from 2 to 9;
(c) a second emulsifier having a HLB value of from 10 to 20;
(d) hydrated silica;
(e) one or more colours; and
(f) one or more fragrances.

In one aspect there is provided a process for the production of a solid cosmetic composition as defined herein the process comprising the steps of:
(i) preparing a first composition comprising
   (a) vegetable butter; and
   (b) a first emulsifier having a HLB value of from 2 to 9;
(ii) preparing a second composition comprising
   (c) a second emulsifier having a HLB value of from 10 to 20;
   (d) one or more inert powders;
   (e) one or more colours; and
   (f) one or more fragrances;
(iii) combining the first composition with the second composition, to form the solid cosmetic composition; wherein the one or more inert powders are present in an amount of from 0.5 to 25 wt. % based on the solid cosmetic composition.

In one aspect there is provided a product obtained or obtainable by a process as described herein.

In one aspect there is provided a cosmetic method comprising contacting the skin of a user with a cosmetic prepared from a solid cosmetic composition as described herein and water.

For ease of reference, these and further aspects of the present invention are now discussed under appropriate section headings. However, the teachings under each section are not necessarily limited to each particular section.

### Advantages

We have identified that by providing a solid cosmetic composition as described herein, a composition is prepared which may be used in at least two ways.

In a first use, the solid cosmetic composition may be deposited in bathing water and will be dispersed in that water. In other words, a bathing oil may be provided which is delivered in solid form. The solid product holds its form and does not readily melt prior to use. By virtue of this property, it is possible to sell the product without unnecessary packaging. The vegetable butter acts as an excellent carrier for oils, colours and other ingredients, which are desirable for their tranquilising and visual properties, while simultaneously possessing excellent emolliating properties of their own. It was found that it was preferable to use soft vegetable butters for the composition, particularly as the melting point of a soft vegetable butter would allow the composition to readily melt and dissipate. For the avoidance of doubt, there is no predefined bathing temperature range for human use, although it is widely accepted that a comfortable bathing temperature is slightly above human body temperature (on average 37° C.) and that an unbearable bathing temperature would be above 47° C.

In addition, we have found that by selecting the particular first emulsifier and second emulsifier required by the present invention, the emulsifier combination is able to emulsify the fats and one or more colours present in the product to produce a pleasant visual effect whereby the colouring and/or fats are effectively dispersed throughout the bathing water.

In a second use, the solid composition may be heated to melt the solid product and to release fragrance from the product. This usage may typically be performed by placing the product in a “wax melt burner”. These “burners” are well-known to those skilled in the art and to users of fragrance products. They typically contain a dish portion in which a solid product is placed and to which heat is applied. This heat may be provided electrically or from a small flame such as a candle. Although these devices are referred to as “burners” the product itself is not combusted. Instead, fragrance in the product which is volatile is released during the heating and provides the desired fragrance release for the user. When the fragrance is no longer required, the heat source is removed and the product solidifies. It may then be reused. The present inventors found that previously when one heated a product which is suitable for cosmetic use, such as a bath oil, numerous problems occurred. In particular, the one or more colours present in the cosmetic product would separate from the vegetable butter during heating and would pool at the bottom of the liquid heated product. This resulted in difficulty in removing the solidified product after cooling from the heating bowl. The solidified product would have a layer of highly concentrated colouring that would stain the hands of the user and other contact surfaces. The solidified product may also be difficult to remove from the bowl, adhering to the bowl and/or breaking up on removal. It was found that by providing a product having both the specific combination of emulsifiers and requiring the presence of the inert powder, these problems could be overcome. In particular, the emulsifiers would assist in the dispersion of the one or more colours within the vegetable butter and the inert powder would be deposited on the bottom of the bowl during the melting of the product and provide a layer between the bowl and the remaining product. This would allow for ease of removal of the solidified product after use.

The present inventors have therefore found that a specific combination of a first emulsifier having a HLB value of from 2 to 9; a second emulsifier having a HLB value of from 10 to 20; and one or more inert powders in an amount of from 0.5 to 25 wt. % based on the solid cosmetic composition, allows for the provision of a solid composition which has both a cosmetic application and a fragrance delivery application. In particular, this specific combination of components allows for the delivery of vegetable butter in bathing water, together with dispersion of colours, to offer both a visually appealing and emollient bath. Furthermore, this specific combination of components allows for the same composition also to be used as a “melt” for the delivery of fragrance upon heating. After solidification this product may be easily removed from the heating bowl. The provision of a product which is cosmetically acceptable and can deliver these dual uses, is highly surprising.

### BRIEF DESCRIPTION OF THE DRAWINGS

The present invention will now be described in further detail by way of example only with reference to the accompanying figures in which:

FIG. 1 is images of the samples of Trial 1;

FIGS. 2A and 2B are images of the samples of Trial 2;

FIG. 3 is images of the samples of Trial 3;

FIG. 4 is images of the samples of Trial 4;

FIG. 5 is images of the samples of Trial 5; and

FIG. 6 is images of the samples of Trial 6.

### DETAILED DESCRIPTION

### Composition

As discussed herein, in one aspect of the present invention, there is provided a solid cosmetic composition comprising
(a) vegetable butter;
(b) a first emulsifier having a HLB value of from 2 to 9;
(c) a second emulsifier having a HLB value of from 10 to 20;
(d) one or more inert powders in an amount of from 0.5 to 25 wt. % based on the solid cosmetic composition;
(e) one or more colours; and
(f) one or more fragrances.

It will be understood by one skilled in the art that the nature of a cosmetic product means that the product is not edible. Thus in a further aspect the present invention provides a non-edible solid cosmetic composition comprising
(a) vegetable butter;
(b) a first emulsifier having a HLB value of from 2 to 9;
(c) a second emulsifier having a HLB value of from 10 to 20;
(d) one or more inert powders in an amount of from 0.5 to 25 wt. % based on the solid cosmetic composition;
(e) one or more colours; and
(f) one or more fragrances.

The solid products of the present invention are compositions which can substantially sustain their physical shape when unsupported by external means, e.g. packaging etc. Thus, they are considered to be solid, solid-like, in solid form or in solid-like form at room temperature. For the avoidance of doubt the solid product must remain substantially solid at up to 30° C.

By solid-like, it is understood that some materials are considered on a day to day basis to be solid, yet over an extremely long period of time, may alter in shape, e.g. amorphous materials such as glass etc. However, they are considered to be solid-like as, for the purpose they fulfil, they are solid.

Due to the solid form of the compositions of the present invention, external packaging is not required to maintain the shape of the composition.

The vegetable butters used in the present invention are triglycerides, which are found to be solid (including solid-like, discussed above) at normal usage temperatures. The term vegetable butter is understood by one skilled in the art and means a triglyceride obtainable from vegetable source which has the consistency of a butter. It is understood by one skilled in the art that a triglyceride obtainable from vegetable source may be a triglyceride obtained from vegetable source which is then subjected to modification provided it remains a triglyceride. For example, a triglyceride obtained from vegetable source may be subjected to hydrogenation to increase the degree of saturation and the solid fat content to provide a vegetable butter. For the avoidance of doubt the vegetable butter is a triglyceride which remains substantially solid at up to 30° C. It will be appreciated however that it is not a requirement that the vegetable butter have a solid fat content of 100% at normal usage temperatures. In a preferred aspect the vegetable butter is a triglyceride having a solid fat content of at least 70%, preferably at least 80%, preferably at least 90%, preferably at least 95%, preferably at least 98%, preferably at least 99% at 25° C.

The vegetable oils used in the present invention are triglycerides, which are found to be liquid at normal usage temperatures. For the avoidance of doubt the vegetable oil is a triglyceride which remains substantially liquid at up to 30° C. It will be appreciated however that it is not a requirement that the vegetable butter have a solid fat content of 0% at normal usage temperatures. In a preferred aspect the vegetable oil is a triglyceride having a solid fat content of no greater than 30%, preferably no greater than 20%, preferably no greater than 10%, preferably no greater than 5%, preferably no greater than 2%, preferably no greater than 1% at 25° C.

In one aspect the composition of the present invention is made by moulding of the product. A typical process comprises the steps of: (i) preparing a first composition comprising (a) vegetable butter; and (b) a first emulsifier having a HLB value of from 2 to 9; (ii) preparing a second composition comprising (c) a second emulsifier having a HLB value of from 10 to 20; (d) one or more inert powders; (e) one or more colours; (f) one or more fragrances;

(iii) combining the first composition with the second composition, wherein the one or more inert powders are present in an amount of from 0.5 to 25 wt. % based on the solid cosmetic composition, placing in a mould and allowing to solidify to form the solid cosmetic composition.

### Vegetable Butter and Vegetable Oil

As discussed herein, the present composition contains vegetable butter. The vegetable butter may be present in any suitable amount. In one aspect, the vegetable butter is present in an amount of from 30 to 80 wt. % based on the solid cosmetic composition. In one aspect, the vegetable butter is present in an amount of from 35 to 80 wt. % based on the solid cosmetic composition. In one aspect, the vegetable butter is present in an amount of from 40 to 80 wt. % based on the solid cosmetic composition. In one aspect, the vegetable butter is present in an amount of from 45 to 80 wt. % based on the solid cosmetic composition. In one aspect, the vegetable butter is present in an amount of from 50 to 80 wt. % based on the solid cosmetic composition. In one aspect, the vegetable butter is present in an amount of from 55 to 80 wt. % based on the solid cosmetic composition. In one aspect, the vegetable butter is present in an amount of from 60 to 80 wt. % based on the solid cosmetic composition. In one aspect, the vegetable butter is present in an amount of from 65 to 80 wt. % based on the solid cosmetic composition. In one aspect, the vegetable butter is present in an amount of from 70 to 80 wt. % based on the solid cosmetic composition. In one aspect, the vegetable butter is present in an amount of from 75 to 80 wt. % based on the solid cosmetic composition.

In one aspect, the vegetable butter is present in an amount of from 30 to 70 wt. % based on the solid cosmetic composition. In one aspect, the vegetable butter is present in an amount of from 35 to 70 wt. % based on the solid cosmetic composition. In one aspect, the vegetable butter is present in an amount of from 40 to 70 wt. % based on the solid cosmetic composition. In one aspect, the vegetable butter is present in an amount of from 45 to 70 wt. % based on the solid cosmetic composition. In one aspect, the vegetable butter is present in an amount of from 50 to 70 wt. % based on the solid cosmetic composition. In one aspect, the vegetable butter is present in an amount of from 55 to 70 wt. % based on the solid cosmetic composition. In one aspect, the vegetable butter is present in an amount of from 60 to 70 wt. % based on the solid cosmetic composition. In one aspect, the vegetable butter is present in an amount of from 65 to 70 wt. % based on the solid cosmetic composition.

The vegetable butter may be selected from cocoa butter, illipe butter, murumuru butter, kokum butter, aloe butter, avocado butter, cupuacu butter, macadamia nut butter, mango butter, olive butter, shea butter, coconut butter, pumpkin seed butter, peanut butter, almond butter, coffee bean butter, refined butter, hemp seed butter, mochacchino butter, pistachio nut butter, shealoe butter and mixtures thereof. In one aspect, the vegetable butter, is selected from cocoa butter, Illipe butter, Kokum butter, Cupuacu butter, Shea butter, and mixtures thereof. In one aspect, the vegetable butter is at least cocoa butter. In one aspect, the vegetable butter consists of cocoa butter.

In one aspect, the solid cosmetic composition may further comprise vegetable oil. If present, the vegetable oil may be present in any suitable amount. If present, the vegetable oil may be present in any suitable amount. In one aspect, the vegetable oil is present in an amount of from 0.1 to 50 wt. % based on the solid cosmetic composition. In one aspect, the vegetable oil is present in an amount of from 0.1 to 40 wt. % based on the solid cosmetic composition. In one aspect, the vegetable oil is present in an amount of from 0.1 to 30 wt. % based on the solid cosmetic composition. In one aspect, the vegetable oil is present in an amount of from 0.1 to 30 wt. % based on the solid cosmetic composition. In one aspect, the vegetable oil is present in an amount of from 0.1 to 20 wt. % based on the solid cosmetic composition. In one aspect, the vegetable oil is present in an amount of from 0.1 to 10 wt. % based on the solid cosmetic composition. In one aspect, the vegetable oil is present in an amount of from 0.1 to 8 wt. % based on the solid cosmetic composition. In one aspect, the vegetable oil is present in an amount of from 0.1 to 6 wt. % based on the solid cosmetic composition. In one aspect, the vegetable oil is present in an amount of from 0.1 to 4 wt. % based on the solid cosmetic composition. In one aspect, the vegetable oil is present in an amount of from 0.1 to 2 wt. % based on the solid cosmetic composition.

In one aspect, the vegetable oil is present in an amount of from 0.5 to 50 wt. % based on the solid cosmetic composition. In one aspect, the vegetable oil is present in an amount of from 0.5 to 40 wt. % based on the solid cosmetic composition. In one aspect, the vegetable oil is present in an amount of from 0.5 to 30 wt. % based on the solid cosmetic composition. In one aspect, the vegetable oil is present in an amount of from 0.5 to 30 wt. % based on the solid cosmetic composition. In one aspect, the vegetable oil is present in an amount of from 0.5 to 20 wt. % based on the solid cosmetic composition. In one aspect, the vegetable oil is present in an amount of from 0.5 to 10 wt. % based on the solid cosmetic composition. In one aspect, the vegetable oil is present in an amount of from 0.5 to 8 wt. % based on the solid cosmetic composition. In one aspect, the vegetable oil is present in an amount of from 0.5 to 6 wt. % based on the solid cosmetic composition. In one aspect, the vegetable oil is present in an amount of from 0.5 to 4 wt. % based on the solid cosmetic composition. In one aspect, the vegetable oil is present in an amount of from 0.5 to 2 wt. % based on the solid cosmetic composition.

In one aspect, the vegetable oil is present in an amount of from 1 to 50 wt. % based on the solid cosmetic composition. In one aspect, the vegetable oil is present in an amount of from 1 to 40 wt. % based on the solid cosmetic composition. In one aspect, the vegetable oil is present in an amount of from 1 to 30 wt. % based on the solid cosmetic composition. In one aspect, the vegetable oil is present in an amount of from 1 to 30 wt. % based on the solid cosmetic composition. In one aspect, the vegetable oil is present in an amount of from 1 to 20 wt. % based on the solid cosmetic composition. In one aspect, the vegetable oil is present in an amount of from 1 to 10 wt. % based on the solid cosmetic composition. In one aspect, the vegetable oil is present in an amount of from 1 to 8 wt. % based on the solid cosmetic composition. In one aspect, the vegetable oil is present in an amount of from 1 to 6 wt. % based on the solid cosmetic composition. In one aspect, the vegetable oil is present in an amount of from 1 to 4 wt. % based on the solid cosmetic composition. In one aspect, the vegetable oil is present in an amount of from 1 to 2 wt. % based on the solid cosmetic composition.

The vegetable oil may be selected from almond oil, jojoba oil, castor oil, olive oil, grape seed oil, argan oil, moringa oil, baobab oil, rose hip oil, kalahari melon oil, brazil nut oil and mixtures thereof. In one aspect, the vegetable oil is selected from almond oil, jojoba oil, and mixtures thereof. In one aspect, the vegetable oil is at least almond oil. In one aspect, the vegetable butter consists of almond oil.

### First Emulsifier

As discussed herein the present composition comprises a first emulsifier having a HLB value of from 2 to 9. As will be understood by one skilled in the art HLB is the hydrophilic-lipophilic balance of a surfactant is a measure of the degree to which it is hydrophilic or lipophilic. The HLB value may be determined by calculating values for the different regions of the molecule, as described by Griffin in Griffin, William C. (1949), “Classification of Surface-Active Agents by ‘HLB’” (PDF), Journal of the Society of Cosmetic Chemists, 1 (5): 311-26 and Griffin, William C. (1954), “Calculation of HLB Values of Non-Ionic Surfactants” (PDF), Journal of the Society of Cosmetic Chemists, 5 (4): 249-56, and by Davies in Davies JT (1957), “A quantitative kinetic theory of emulsion type, I. Physical chemistry of the emulsifying agent” (PDF), Gas/Liquid and Liquid/Liquid Interface, Proceedings of the International Congress of Surface Activity, pp. 426-38. HLB value may be determined in accordance with the industry standard text book, namely “The HLB SYSTEM, a time-saving guide to emulsifier selection” ICI Americas Inc., Published 1976 and Revised, March, 1980. The HLB values of the emulsifiers described herein were determined in accordance with this standard method.

In one aspect the first emulsifier has a HLB value of from 3 to 9. In one aspect the first emulsifier has a HLB value of from 4 to 9. In one aspect the first emulsifier has a HLB value of from 4.3 to 9.

The first emulsifier may be selected from any suitable emulsifier having a HLB value of from 2 to 9. In one aspect the first emulsifier is an emulsifier having a HLB value of from 2 to 9 selected from mono and diglycerydes of fatty acid including glyceryl stearate and glyceryl oleate; fatty acid esters of C12-C22 fatty alcohols including fatty acid esters of cetyl alcohol and fatty acid esters of stearoyl alcohol, mixtures of fatty acid esters of cetyl alcohol and fatty acid esters of stearoyl alcohol, mixtures of fatty acid esters of cetyl alcohol and fatty acid esters of stearoyl alcohol wherein the fatty acids are derived from olive oil (such as cetearly olivate), fatty acid esters of sorbitol including sorbitan oleate, fatty acid esters of sorbitol wherein the fatty acids are derived from olive oil (such as sorbitan olivate or cetearyl olivate), and mixtures thereof. In one aspect the first emulsifier is an emulsifier having a HLB value of from 2 to 9 selected from mono and diglycerydes of fatty acid, fatty acid esters of C12-C22 fatty alcohols, fatty acid esters of sorbitol, and mixtures thereof.

The first emulsifier may be present in any suitable amount. In one aspect, the first emulsifier is present in an amount of from 2 to 30 wt. % based on the solid cosmetic composition. In one aspect, the first emulsifier is present in an amount of from 2 to 25 wt. % based on the solid cosmetic composition. In one aspect, the first emulsifier is present in an amount of from 2 to 20 wt. % based on the solid cosmetic composition. In one aspect, the first emulsifier is present in an amount of from 2 to 18 wt. % based on the solid cosmetic composition. In one aspect, the first emulsifier is present in an amount of from 2 to 16 wt. % based on the solid cosmetic composition. In one aspect, the first emulsifier is present in an amount of from 2 to 14 wt. % based on the solid cosmetic composition. In one aspect, the first emulsifier is present in an amount of from 2 to 12 wt. % based on the solid cosmetic composition. In one aspect, the first emulsifier is present in an amount of from 2 to 10 wt. % based on the solid cosmetic composition. In one aspect, the first emulsifier is present in an amount of from 2 to 8 wt. % based on the solid cosmetic composition. In one aspect, the first emulsifier is present in an amount of from 2 to 7 wt. % based on the solid cosmetic composition.

In one aspect, the first emulsifier is present in an amount of from 3 to 30 wt. % based on the solid cosmetic composition. In one aspect, the first emulsifier is present in an amount of from 3 to 25 wt. % based on the solid cosmetic composition. In one aspect, the first emulsifier is present in an amount of from 3 to 20 wt. % based on the solid cosmetic composition. In one aspect, the first emulsifier is present in an amount of from 4 to 20 wt. % based on the solid cosmetic composition. In one aspect, the first emulsifier is present in an amount of from 5 to 20 wt. % based on the solid cosmetic composition. In one aspect, the first emulsifier is present in an amount of from 6 to 20 wt. % based on the solid cosmetic composition.

In one aspect, the first emulsifier is present in an amount of from 5 to 30 wt. % based on the solid cosmetic composition. In one aspect, the first emulsifier is present in an amount of from 5 to 25 wt. % based on the solid cosmetic composition. In one aspect, the first emulsifier is present in an amount of from 5 to 20 wt. % based on the solid cosmetic composition. In one aspect, the first emulsifier is present in an amount of from 5 to 18 wt. % based on the solid cosmetic composition. In one aspect, the first emulsifier is present in an amount of from 5 to 16 wt. % based on the solid cosmetic composition. In one aspect, the first emulsifier is present in an amount of from 5 to 14 wt. % based on the solid cosmetic composition. In one aspect, the first emulsifier is present in an amount of from 5 to 12 wt. % based on the solid cosmetic composition. In one aspect, the first emulsifier is present in an amount of from 5 to 10 wt. % based on the solid cosmetic composition.

In one aspect, the first emulsifier is present in an amount of from 5 to 30 wt. % based on the solid cosmetic composition. In one aspect, the first emulsifier is present in an amount of from 7 to 30 wt. % based on the solid cosmetic composition. In one aspect, the first emulsifier is present in an amount of from 10 to 30 wt. % based on the solid cosmetic composition. In one aspect, the first emulsifier is present in an amount of from 10 to 25 wt. % based on the solid cosmetic composition. In one aspect, the first emulsifier is present in an amount of from 10 to 20 wt. % based on the solid cosmetic composition. In one aspect, the first emulsifier is present in an amount of from 12 to 18 wt. % based on the solid cosmetic composition.

### Second Emulsifier

As discussed herein the present composition comprises a second emulsifier having a HLB value of from 10 to 20. In one aspect, the second emulsifier has a HLB value of from 10 to 19. In one aspect, the second emulsifier has a HLB value of from 10 to 18. In one aspect, the second emulsifier has a HLB value of from 10 to 13. In one aspect, the second emulsifier has a HLB value of from 10 to 17. In one aspect, the second emulsifier has a HLB value of from 10 to 16. In one aspect, the second emulsifier has a HLB value of from 10 to 15. In one aspect, the second emulsifier has a HLB value of from 10 to 14. In one aspect, the second emulsifier has a HLB value of from 10 to 13. In one aspect, the second emulsifier has a HLB value of from 10 to 12.

The second emulsifier may be selected from any suitable emulsifier having a HLB value of from 10 to 20. In one aspect, the second emulsifier is an emulsifier having a HLB value of from 10 to 20 selected from fatty acid esters of polyethylene glycol, such as fatty acid esters of polyethylene glycol wherein the fatty acids are derived from coconut oil (including PEG 7), fatty acid esters of polyglycerol including fatty acid esters of polyglycerol and oleic acid (such as polyglyceryl 10 oleate), and mixtures thereof. In one aspect, the second emulsifier is an emulsifier having a HLB value of from 10 to 20 selected from fatty acid esters of polyethylene glycol, fatty acid esters of polyglycerol, and mixtures thereof.

The second emulsifier may be present in any suitable amount. In one aspect, the second emulsifier is present in an amount of from 2 to 30 wt. % based on the solid cosmetic composition. In one aspect, the second emulsifier is present in an amount of from 2 to 25 wt. % based on the solid cosmetic composition. In one aspect, the second emulsifier is present in an amount of from 2 to 20 wt. % based on the solid cosmetic composition. In one aspect, the second emulsifier is present in an amount of from 2 to 18 wt. % based on the solid cosmetic composition. In one aspect, the second emulsifier is present in an amount of from 2 to 16 wt. % based on the solid cosmetic composition. In one aspect, the second emulsifier is present in an amount of from 2 to 14 wt. % based on the solid cosmetic composition. In one aspect, the second emulsifier is present in an amount of from 2 to 12 wt. % based on the solid cosmetic composition. In one aspect, the second emulsifier is present in an amount of from 2 to 10 wt. % based on the solid cosmetic composition.

In one aspect, the second emulsifier is present in an amount of from 3 to 30 wt. % based on the solid cosmetic composition. In one aspect, the second emulsifier is present in an amount of from 3 to 25 wt. % based on the solid cosmetic composition. In one aspect, the second emulsifier is present in an amount of from 3 to 20 wt. % based on the solid cosmetic composition. In one aspect, the second emulsifier is present in an amount of from 4 to 20 wt. % based on the solid cosmetic composition. In one aspect, the second emulsifier is present in an amount of from 5 to 20 wt. % based on the solid cosmetic composition. In one aspect, the second emulsifier is present in an amount of from 6 to 20 wt. % based on the solid cosmetic composition. In one aspect, the second emulsifier is present in an amount of from 7 to 20 wt. % based on the solid cosmetic composition.

In one aspect, the second emulsifier is present in an amount of from 5 to 30 wt. % based on the solid cosmetic composition. In one aspect, the second emulsifier is present in an amount of from 5 to 25 wt. % based on the solid cosmetic composition. In one aspect, the second emulsifier is present in an amount of from 5 to 20 wt. % based on the solid cosmetic composition. In one aspect, the second emulsifier is present in an amount of from 5 to 18 wt. % based on the solid cosmetic composition. In one aspect, the second emulsifier is present in an amount of from 5 to 16 wt. % based on the solid cosmetic composition. In one aspect, the second emulsifier is present in an amount of from 5 to 14 wt. % based on the solid cosmetic composition. In one aspect, the second emulsifier is present in an amount of from 5 to 12 wt. % based on the solid cosmetic composition. In one aspect, the second emulsifier is present in an amount of from 5 to 10 wt. % based on the solid cosmetic composition.

In one aspect, the second emulsifier is present in an amount of from 5 to 30 wt. % based on the solid cosmetic composition. In one aspect, the second emulsifier is present in an amount of from 7 to 30 wt. % based on the solid cosmetic composition. In one aspect, the second emulsifier is present in an amount of from 10 to 30 wt. % based on the solid cosmetic composition. In one aspect, the second emulsifier is present in an amount of from 10 to 25 wt. % based on the solid cosmetic composition. In one aspect, the second emulsifier is present in an amount of from 10 to 20 wt. % based on the solid cosmetic composition. In one aspect, the second emulsifier is present in an amount of from 12 to 18 wt. % based on the solid cosmetic composition.

### Inert Powder

The composition comprises (d) one or more inert powders in an amount of from 0.5 to 25 wt. % based on the solid cosmetic composition.

The one or more inert powders may be selected from all suitable inert powders. In one aspect the one or more inert powders is selected from silicates (such as phyllosilicates, such as mica, such as fluorphlogopite, and such as synthetic fluorphlogopite); titanium dioxide; tin oxide; zinc oxide; silicas (such as glasses, such as borosilicates and such as calcium sodium borosilicate); zeolites (including zeolite containing polysulfides, such as sodium aluminosilicate and such as Al₆Na₆O₂₄S₈Si₆); hydrated silica; metal carbonates (such as sodium carbonate, calcium carbonate, sodium bicarbonate and mixtures thereof); silicates (such as layered silicates and such as kaolin); and mixtures thereof.

In one aspect the one or more inert powders is selected from phyllosilicates (such as mica, such as fluorphlogopite, and such as synthetic fluorphlogopite); titanium dioxide; tin oxide; zinc oxide; borosilicates (such as calcium sodium borosilicate); zeolites (such as sodium aluminosilicate and such as Al₆Na₆O₂₄S₈Si₆); hydrated silica; metal carbonates (such as sodium carbonate, calcium carbonate, sodium bicarbonate and mixtures thereof); layered silicates (such as kaolin); and mixtures thereof.

In one aspect the one or more inert powders is selected from mica (such as fluorphlogopite, and such as synthetic fluorphlogopite); titanium dioxide; tin oxide; zinc oxide; calcium sodium borosilicate; sodium aluminosilicate (such as Al₆Na₆O₂₄S₈Si₆); hydrated silica; sodium carbonate; calcium carbonate; sodium bicarbonate; kaolin; and mixtures thereof.

In one aspect the one or more inert powders is selected from synthetic fluorphlogopite; titanium dioxide; zinc oxide; sodium aluminosilicate; hydrated silica; and mixtures thereof.

In one aspect the one or more inert powders comprises at least hydrated silica. In one aspect the one or more inert powders consists of hydrated silica.

As noted herein, the composition comprises (d) one or more inert powders in an amount of from 0.5 to 25 wt. % based on the solid cosmetic composition. In one aspect, the one or more inert powders are present (such as silica is present) in an amount of from 1 to 25 wt. % based on the solid cosmetic composition. In one aspect, the one or more inert powders are present (such as silica is present) in an amount of from 2 to 25 wt. % based on the solid cosmetic composition. In one aspect, the one or more inert powders are present (such as silica is present) in an amount of from 3 to 25 wt. % based on the solid cosmetic composition. In one aspect, the one or more inert powders are present (such as silica is present) in an amount of from 4 to 25 wt. % based on the solid cosmetic composition. In one aspect, the one or more inert powders are present (such as silica is present) in an amount of from 5 to 25 wt. % based on the solid cosmetic composition. In one aspect, the one or more inert powders are present (such as silica is present) in an amount of from 7 to 25 wt. % based on the solid cosmetic composition. In one aspect, the one or more inert powders are present (such as silica is present) in an amount of from 10 to 25 wt. % based on the solid cosmetic composition.

In one aspect, the one or more inert powders are present (such as silica is present) in an amount of from 0.5 to 20 wt. % based on the solid cosmetic composition. In one aspect, the one or more inert powders are present (such as silica is present) in an amount of from 1 to 20 wt. % based on the solid cosmetic composition. In one aspect, the one or more inert powders are present (such as silica is present) in an amount of from 2 to 20 wt. % based on the solid cosmetic composition. In one aspect, the one or more inert powders are present (such as silica is present) in an amount of from 3 to 20 wt. % based on the solid cosmetic composition. In one aspect, the one or more inert powders are present (such as silica is present) in an amount of from 4 to 20 wt. % based on the solid cosmetic composition. In one aspect, the one or more inert powders are present (such as silica is present) in an amount of from 5 to 20 wt. % based on the solid cosmetic composition. In one aspect, the one or more inert powders are present (such as silica is present) in an amount of from 7 to 20 wt. % based on the solid cosmetic composition. In one aspect, the one or more inert powders are present (such as silica is present) in an amount of from 10 to 20 wt. % based on the solid cosmetic composition.

In one aspect, the one or more inert powders are present (such as silica is present) in an amount of from 0.5 to 15 wt. % based on the solid cosmetic composition. In one aspect, the one or more inert powders are present (such as silica is present) in an amount of from 1 to 15 wt. % based on the solid cosmetic composition. In one aspect, the one or more inert powders are present (such as silica is present) in an amount of from 2 to 15 wt. % based on the solid cosmetic composition. In one aspect, the one or more inert powders are present (such as silica is present) in an amount of from 3 to 15 wt. % based on the solid cosmetic composition. In one aspect, the one or more inert powders are present (such as silica is present) in an amount of from 4 to 15 wt. % based on the solid cosmetic composition. In one aspect, the one or more inert powders are present (such as silica is present) in an amount of from 5 to 15 wt. % based on the solid cosmetic composition. In one aspect, the one or more inert powders are present (such as silica is present) in an amount of from 7 to 15 wt. % based on the solid cosmetic composition. In one aspect, the one or more inert powders are present (such as silica is present) in an amount of from 10 to 15 wt. % based on the solid cosmetic composition.

In one aspect, the one or more inert powders are present (such as silica is present) in an amount of from 0.5 to 22 wt. % based on the solid cosmetic composition. In one aspect, the one or more inert powders are present (such as silica is present) in an amount of from 0.5 to 18 wt. % based on the solid cosmetic composition. In one aspect, the one or more inert powders are present (such as silica is present) in an amount of from 0.5 to 16 wt. % based on the solid cosmetic composition. In one aspect, the one or more inert powders are present (such as silica is present) in an amount of from 0.5 to 14 wt. % based on the solid cosmetic composition. In one aspect, the one or more inert powders are present (such as silica is present) in an amount of from 0.5 to 12 wt. % based on the solid cosmetic composition. In one aspect, the one or more inert powders are present (such as silica is present) in an amount of from 0.5 to 10 wt. % based on the solid cosmetic composition. In one aspect, the one or more inert powders are present (such as silica is present) in an amount of from 0.5 to 8 wt. % based on the solid cosmetic composition. In one aspect, the one or more inert powders are present (such as silica is present) in an amount of from 0.5 to 6 wt. % based on the solid cosmetic composition. In one aspect, the one or more inert powders are present (such as silica is present) in an amount of from 0.5 to 4 wt. % based on the solid cosmetic composition. In one aspect, the one or more inert powders are present (such as silica is present) in an amount of from 0.5 to 2 wt. % based on the solid cosmetic composition.

In one aspect, the one or more inert powders are present (such as silica is present) in an amount of from 1 to 25 wt. % based on the solid cosmetic composition. In one aspect, the one or more inert powders are present (such as silica is present) in an amount of from 1 to 22 wt. % based on the solid cosmetic composition. In one aspect, the one or more inert powders are present (such as silica is present) in an amount of from 1 to 20 wt. % based on the solid cosmetic composition. In one aspect, the one or more inert powders are present (such as silica is present) in an amount of from 1 to 18 wt. % based on the solid cosmetic composition. In one aspect, the one or more inert powders are present (such as silica is present) in an amount of from 1 to 16 wt. % based on the solid cosmetic composition. In one aspect, the one or more inert powders are present (such as silica is present) in an amount of from 1 to 14 wt. % based on the solid cosmetic composition. In one aspect, the one or more inert powders are present (such as silica is present) in an amount of from 1 to 12 wt. % based on the solid cosmetic composition. In one aspect, the one or more inert powders are present (such as silica is present) in an amount of from 1 to 10 wt. % based on the solid cosmetic composition. In one aspect, the one or more inert powders are present (such as silica is present) in an amount of from 1 to 8 wt. % based on the solid cosmetic composition. In one aspect, the one or more inert powders are present (such as silica is present) in an amount of from 1 to 6 wt. % based on the solid cosmetic composition. In one aspect, the one or more inert powders are present (such as silica is present) in an amount of from 1 to 4 wt. % based on the solid cosmetic composition.

As noted herein, in one aspect the one or more inert powders may comprise at least hydrated silica. In one aspect the one or more inert powders consists of hydrated silica. We have found that hydrated silica is particularly advantageous as it modifies the viscosity of the vegetable butter (fat) when the product is melted in use. In particular, the hydrated silica is found to thicken the liquid fat and thereby reduce the settling out of colours and other materials prone to sedimentation. These colours and other materials are more easily suspended in systems comprising hydrated silica and/or increased amounts of colours and other materials prone to sedimentation may be provided in compositions comprising hydrated silica owing to this advantageous property of hydrated silica. We have also found that hydrated silica may gather at the bottom of the vegetable butter (liquid fat) when melted in use and provides a localised area of high viscosity at the bottom of the liquid fat. This reduces the tendency of the composition to adhere to the surface of the burner on cooling and improves the ease of removal of the solidified composition from the burner. Thus, in one aspect there is provided a solid cosmetic composition comprising
(a) vegetable butter;
(b) a first emulsifier having a HLB value of from 2 to 9;
(c) a second emulsifier having a HLB value of from 10 to 20;
(d) hydrated silica in an amount of from 0.5 to 25 wt. % based on the solid cosmetic composition;
(e) one or more colours; and
(f) one or more fragrances.

In a further broad aspect there is provided a solid cosmetic composition comprising
(a) vegetable butter;
(b) a first emulsifier having a HLB value of from 2 to 9;
(c) a second emulsifier having a HLB value of from 10 to 20;
(d) hydrated silica;
(e) one or more colours; and
(f) one or more fragrances.

### Colour

The solid cosmetic composition comprises (e) one or more colours. The one or more colours may be a water soluble colour, oil soluble colour, and mixtures thereof. In one aspect the colour is at least one water soluble colour. In one aspect the colour is at least one oil soluble colour. In one aspect the colour is a mixture of at least one water soluble colour and at least one oil soluble colour.

The one or more colours may be present in any suitable amount. In one aspect the composition contains colour in an amount of from 0.001 to 5 wt. % based on the solid cosmetic composition. In one aspect the composition contains colour in an amount of from 0.001 to 2 wt. % based on the solid cosmetic composition. In one aspect the composition contains colour in an amount of from 0.001 to 1 wt. % based on the solid cosmetic composition. In one aspect the composition contains colour in an amount of from 0.001 to 0.8 wt. % based on the solid cosmetic composition. In one aspect the composition contains colour in an amount of from 0.001 to 0.6 wt. % based on the solid cosmetic composition. In one aspect the composition contains colour in an amount of from 0.001 to 0.5 wt. % based on the solid cosmetic composition. In one aspect the composition contains colour in an amount of from 0.001 to 0.4 wt. % based on the solid cosmetic composition. In one aspect the composition contains colour in an amount of from 0.001 to 0.2 wt. % based on the solid cosmetic composition. In one aspect the composition contains colour in an amount of from 0.001 to 0.1 wt. % based on the solid cosmetic composition.

In one aspect the composition contains colour in an amount of from 0.01 to 5 wt. % based on the solid cosmetic composition. In one aspect the composition contains colour in an amount of from 0.01 to 2 wt. % based on the solid cosmetic composition. In one aspect the composition contains colour in an amount of from 0.01 to 1 wt. % based on the solid cosmetic composition. In one aspect the composition contains colour in an amount of from 0.01 to 0.8 wt. % based on the solid cosmetic composition. In one aspect the composition contains colour in an amount of from 0.01 to 0.6 wt. % based on the solid cosmetic composition. In one aspect the composition contains colour in an amount of from 0.01 to 0.5 wt. % based on the solid cosmetic composition. In one aspect the composition contains colour in an amount of from 0.01 to 0.4 wt. % based on the solid cosmetic composition. In one aspect the composition contains colour in an amount of from 0.01 to 0.2 wt. % based on the solid cosmetic composition. In one aspect the composition contains colour in an amount of from 0.01 to 0.1 wt. % based on the solid cosmetic composition.

In one aspect the composition contains colour in an amount of from 0.02 to 5 wt. % based on the solid cosmetic composition. In one aspect the composition contains colour in an amount of from 0.02 to 2 wt. % based on the solid cosmetic composition. In one aspect the composition contains colour in an amount of from 0.02 to 1 wt. % based on the solid cosmetic composition. In one aspect the composition contains colour in an amount of from 0.02 to 0.8 wt. % based on the solid cosmetic composition. In one aspect the composition contains colour in an amount of from 0.02 to 0.6 wt. % based on the solid cosmetic composition. In one aspect the composition contains colour in an amount of from 0.02 to 0.5 wt. % based on the solid cosmetic composition. In one aspect the composition contains colour in an amount of from 0.02 to 0.4 wt. % based on the solid cosmetic composition. In one aspect the composition contains colour in an amount of from 0.02 to 0.2 wt. % based on the solid cosmetic composition. In one aspect the composition contains colour in an amount of from 0.02 to 0.1 wt. % based on the solid cosmetic composition.

### Fragrance

The composition of the present invention comprises a fragrance. Preferably the fragrance is selected from essential oils. The essential oils may be selected based on the fragrance desired, skin type to be treated and other effects desired based on the well-known properties of essential oils. The addition of essential oils, when taken in to the nose, are known to alter mood. For example, essential oils are known to create effects of drowsiness or stimulating the senses. Many well documented effects can be achieved by the use of essential oils.

In one embodiment, the one or more essential oils present in the solid product are selected from Tarragon, Lemon myrtle, Jasmin, Ylang ylang, Labdunum, Lemongrass, Rose otto, Grapefruit, Patchouli, Rosemary, Armois, Lemon, Neroli, Sweet violet, Lavender, Orange 50 fold, Vanilla, Peppermint, Benzoin, Hydrangia, Litsea Cubeba, Cardamon, Tonka, and Chamomile blue. In one embodiment, the one or more essential oils present in the solid product are selected from Tarragon, Lemon myrtle, Labdunum, and Lemon.

The fragrance may be present in any suitable amount. In one aspect, the fragrance is present in an amount of from 0.01 to 10 wt. % based on the solid cosmetic composition. In one aspect, the fragrance is present in an amount of from 0.01 to 8 wt. % based on the solid cosmetic composition. In one aspect, the fragrance is present in an amount of from 0.01 to 6 wt. % based on the solid cosmetic composition. In one aspect, the fragrance is present in an amount of from 0.01 to 4 wt. % based on the solid cosmetic composition. In one aspect, the fragrance is present in an amount of from 0.01 to 3 wt. % based on the solid cosmetic composition. In one aspect, the fragrance is present in an amount of from 0.01 to 2 wt. % based on the solid cosmetic composition. In one aspect, the fragrance is present in an amount of from 0.01 to 1 wt. % based on the solid cosmetic composition.

In one aspect, the fragrance is present in an amount of from 0.1 to 10 wt. % based on the solid cosmetic composition. In one aspect, the fragrance is present in an amount of from 0.1 to 8 wt. % based on the solid cosmetic composition. In one aspect, the fragrance is present in an amount of from 0.1 to 6 wt. % based on the solid cosmetic composition. In one aspect, the fragrance is present in an amount of from 0.1 to 4 wt. % based on the solid cosmetic composition. In one aspect, the fragrance is present in an amount of from 0.1 to 3 wt. % based on the solid cosmetic composition. In one aspect, the fragrance is present in an amount of from 0.1 to 2 wt. % based on the solid cosmetic composition. In one aspect, the fragrance is present in an amount of from 0.1 to 1 wt. % based on the solid cosmetic composition.

In one aspect, the fragrance is present in an amount of from 0.02 to 10 wt. % based on the solid cosmetic composition. In one aspect, the fragrance is present in an amount of from 0.03 to 10 wt. % based on the solid cosmetic composition. In one aspect, the fragrance is present in an amount of from 0.05 to 10 wt. % based on the solid cosmetic composition.

In one aspect, the fragrance is present in an amount of from 0.07 to 10 wt. % based on the solid cosmetic composition. In one aspect, the fragrance is present in an amount of from 0.2 to 10 wt. % based on the solid cosmetic composition. In one aspect, the fragrance is present in an amount of from 0.3 to 10 wt. % based on the solid cosmetic composition. In one aspect, the fragrance is present in an amount of from 0.5 to 10 wt. % based on the solid cosmetic composition. In one aspect, the fragrance is present in an amount of from 0.7 to 10 wt. % based on the solid cosmetic composition. In one aspect, the fragrance is present in an amount of from 1 to 10 wt. % based on the solid cosmetic composition. In one aspect, the fragrance is present in an amount of from 1.2 to 10 wt. % based on the solid cosmetic composition. In one aspect, the fragrance is present in an amount of from 1.5 to 10 wt. % based on the solid cosmetic composition. In one aspect, the fragrance is present in an amount of from 1.7 to 10 wt. % based on the solid cosmetic composition. In one aspect, the fragrance is present in an amount of from 2 to 10 wt. % based on the solid cosmetic composition. In one aspect, the fragrance is present in an amount of from 3 to 10 wt. % based on the solid cosmetic composition. In one aspect, the fragrance is present in an amount of from 4 to 10 wt. % based on the solid cosmetic composition. In one aspect, the fragrance is present in an amount of from 5 to 10 wt. % based on the solid cosmetic composition.

### Additional Components

The solid product of the present invention may also comprise one or more cosmetically acceptable additives. The person skilled in the art is aware of a range of cosmetically acceptable additives which are suitable for incorporation into such compositions, for example, binders, fillers, opacifiers, decorative items, organic pigments, inorganic pigments, and mixtures thereof.

Fruit and herb extracts and juices are all compatible with the composition. In one embodiment, the cosmetically acceptable additives are selected from the group consisting of vitamins, clays, decorative articles, and mixtures thereof.

Vitamins, particularly B, C and E are very beneficial for the skin. Vitamin rich ingredients such as Wheatgerm oil can also be used to deliver vitamins on to the skin. In a one embodiment, the vitamins are selected from vitamin B, vitamin C, vitamin E and mixtures thereof. It will be appreciated by one skilled in the art that the vitamin may be provided from any suitable source. For example, the vitamin(s) may be provided from a synthetic source or from incorporation into the solid product of a material, such as a natural material, that has a high vitamin content.

The ingredients in the present invention do not require cosmetic preservatives. The use of cosmetic preservatives can increase the potential to irritate the skin.

The decorative items which may be present in the solid product include items such as glitter, paper such as rice paper, sequins, dried or fresh flowers, herbs, vegetables, parts thereof or mixtures thereof. Other enhancing materials may also be incorporated

Further preferred additive materials include vegetable oils, chocolate, herbs and spices, cosmetic colours (e.g. paprika, gardenia extract, D&C red no. 30), beans (e.g. aduki), fruit, fresh or dried (e.g. banana, avocado, mango, papaya, kiwi, raspberry, strawberry, blueberries, grapes, tomato, asparagus, or cucumber), honey, glycerin, cosmetic glitter, other vegetable butters (e.g. mango, avocado), clays (e.g. kaolin), starches (e.g. corn starch), popping candy and mixtures thereof.

The above ranges provide preferred amounts of each of the components. Each of these ranges may be taken alone or combined with one or more other component ranges to provide a preferred aspect of the invention.

In some embodiments, water may be present in an amount of no greater than about 10% by weight of the solid cosmetic composition. In some embodiments, water may be present in an amount of no greater than about 8% by weight of the solid cosmetic composition. In some embodiments, water may be present in an amount of no greater than about 7% by weight of the solid cosmetic composition. In some embodiments, water may be present in an amount of no greater than about 6% by weight of the solid cosmetic composition. In some embodiments, water may be present in an amount of no greater than about 5% by weight of the solid cosmetic composition. In some embodiments, water may be present in an amount of no greater than about 3% by weight of the solid cosmetic composition. In some embodiments, water may be present in an amount of no greater than about 2.5% by weight of the solid cosmetic composition. In some embodiments, water may be present in an amount of no greater than about 2% by weight of the solid cosmetic composition.

In one aspect the solid composition of the present invention may also comprise one or more waxes. We have found that when the composition comprises vegetable butter in low amounts, the presence of a wax improves the solidity of the product. Thus, in one aspect the composition further comprises a wax. In one aspect the one or more waxes are selected from carnauba wax, candelilla, Japan wax, beeswax, rapeseed wax, sunflower wax, orange peel wax, rose wax, paraffin wax, microcrystalline wax, ozocerite wax and mixtures thereof.

### Process

As discussed herein, the invention provides a process for the production of a solid cosmetic composition as defined herein the process comprising the steps of:
(i) preparing a first composition comprising
   (a) vegetable butter; and
   (b) a first emulsifier having a HLB value of from 2 to 9;
(ii) preparing a second composition comprising
   (c) a second emulsifier having a HLB value of from 10 to 20;
   (d) one or more inert powders;
   (e) one or more colours; and
   (f) one or more fragrances;
(iii) combining the first composition with the second composition, to form the solid cosmetic composition; wherein the one or more inert powders are present in an amount of from 0.5 to 25 wt. % based on the solid cosmetic composition.

As discussed herein the composition of the present invention may be made by moulding of the product. A typical process comprises the steps of: (i) preparing a first composition comprising (a) vegetable butter; and (b) a first emulsifier having a HLB value of from 2 to 9; (ii) preparing a second composition comprising (c) a second emulsifier having a HLB value of from 10 to 20; (d) one or more inert powders; (e) one or more colours; (f) one or more fragrances; (iii) combining the first composition with the second composition, wherein the one or more inert powders are present in an amount of from 0.5 to 25 wt. % based on the solid cosmetic composition, placing in a mould and allowing to solidify to form the solid cosmetic composition.

The shape of the solid products of the present invention is not limited. It may be that the solid products are provided with a shape which would be aesthetically pleasing and/or which aids in the use of the product. For example, it may be that the solid product is produced in such a manner so that it solidifies in a shape which is ergonomically acceptable to the user. Products which are approximately spherical, cuboidal or cylindrical are each envisaged. In one aspect, the product may be formed into a shape which allows for easy usage of the product. In one aspect the product is formed into a single piece which fits in a heater and/or provides sufficient product for a single bath. In one aspect the product is formed into a shape which allows for a single piece to be removed from an overall larger piece of solid. As a non-limiting example, a bar may be provided with indentations or grooves, which allow for a suitably sized piece to be ‘snapped’ from the larger bar.

The present invention also provides a product obtained or obtainable by a process as described herein.

### EXAMPLE

The invention will now be described with reference to the following non-limiting examples.

| Trial | | | Trade | |
|---|---|---|---|---|
| Number | Sample | Inert Powder | Name | Notes |
| 1 | 40022 | Synthetic | TW Silver | |
| | | fluorphlogopite, | | |
| | | titanium dioxide | | |
| | | and tin | | |
| | | oxide. (5-60 μm) | | |
| 2 | 10248 | Synthetic | Synafil | |
| | | fluorphlogopite | S525 | |
| | | (5-25 μm) | | |
| 3 | 13415 | Titanium dioxide | TiO2 | (standard grade, not |
| | | | | treated or microfine) |
| 4 | 10663 | Aluminosilicate | Ultramarine | |
| | | (Al₆Na₈O₂₄S₃Si₆) | Blue CI | |
| | | | 77007 | |
| 5 | 13181 | Hydrated silica | | Median 10 micron |
| 6 | 10777 | Zinc oxide | | Typically 2 micron |

Each of the six inert powders were tested at amounts of 0.1 wt %, 0.5 wt %, 1 wt, 5 wt %, 10 wt % and 20 wt % based on the solid cosmetic composition. The details of each of these formulations is provided below.

0.1 wt. % Inert Powder

| | Formula wt. % | Raw Material Type |
|---|---|---|
| A | 63.95 | Cocoa Butter Org FT DR |
| | 15.00 | Glyceryl stearate SE |
| B | 16.90 | PEG-7 Glyceryl Cocoate |
| | 0.050 | CI42090 FD&C Blue No 1 |
| | 4.00 | Fragrance oil |
| | 0.10 | Inert powder |
| | 100.00 | |

0.5 wt. % Inert Powder

| | Formula wt. % | Raw Material Type |
|---|---|---|
| A | 63.95 | Cocoa Butter Org FT DR |
| | 15.00 | Glyceryl stearate SE |
| B | 16.50 | PEG-7 Glyceryl Cocoate |
| | 0.050 | CI42090 FD&C Blue No 1 |
| | 4.00 | Fragrance oil |
| | 0.50 | Inert powder |
| | 100.00 | |

1 wt. % Inert Powder

| | Formula wt. % | Raw Material Type |
|---|---|---|
| A | 63.95 | Cocoa Butter Org FT DR |
| | 15.00 | Glyceryl stearate SE |
| B | 16.00 | PEG-7 Glyceryl Cocoate |
| | 0.050 | CI42090 FD&C Blue No 1 |
| | 4.00 | Fragrance oil |
| | 1.00 | Inert powder |
| | 100.00 | |

5 wt. % Inert Powder

| | Formula wt. % | Raw Material Type |
|---|---|---|
| A | 60.95 | Cocoa Butter Org FT DR |
| | 15.00 | Glyceryl stearate SE |
| B | 15.00 | PEG-7 Glyceryl Cocoate |
| | 0.050 | CI42090 FD&C Blue No 1 |
| | 4.00 | Fragrance oil |
| | 5.00 | Inert powder |
| | 100.00 | |

10 wt. % Inert Powder

| | Formula % | Raw Material Type |
|---|---|---|
| A | 55.95 | Cocoa Butter Org FT DR |
| | 15.00 | Glyceryl stearate SE |
| B | 15.00 | PEG-7 Glyceryl Cocoate |
| | 0.050 | CI42090 FD&C Blue No 1 |
| | 4.00 | Fragrance oil |
| | 10.00 | Inert powder |
| | 100.00 | |

20 wt. % Inert Powder

| | Formula % | Raw Material Type |
|---|---|---|
| A | 45.95 | Cocoa Butter Org FT DR |
| | 15.00 | Glyceryl stearate SE |
| B | 15.00 | PEG-7 Glyceryl Cocoate |
| | 0.050 | CI42090 FD&C Blue No 1 |
| | 4.00 | Fragrance oil |
| | 20.00 | Inert powder |
| | 100.00 | |

The following method was used in each case
1. Warm A gently to 60° C. to melt
2. Temper the mixture (cool down the butters and oils to 27° C. and then heat them back up to 37° C. before cooling again)
3. Mix B and add to A when below 37° C.
4. Pour when the mix is below 25° C. or when the mix is opaque

Each of the prepared products was placed in a “wax burner” and heated until fully molten using a candle placed below the bowl of the wax burner. The candle was then extinguished and the products allowed to solidify. Once the products had fully cooled, the solidified product was removed from the bowl of the wax burner. The observed results of the trials were as follows. The images of the products are also show in the figures.

### Trial 1

0.1% not enough
1% just enough
5% very good
10% very good
20% works

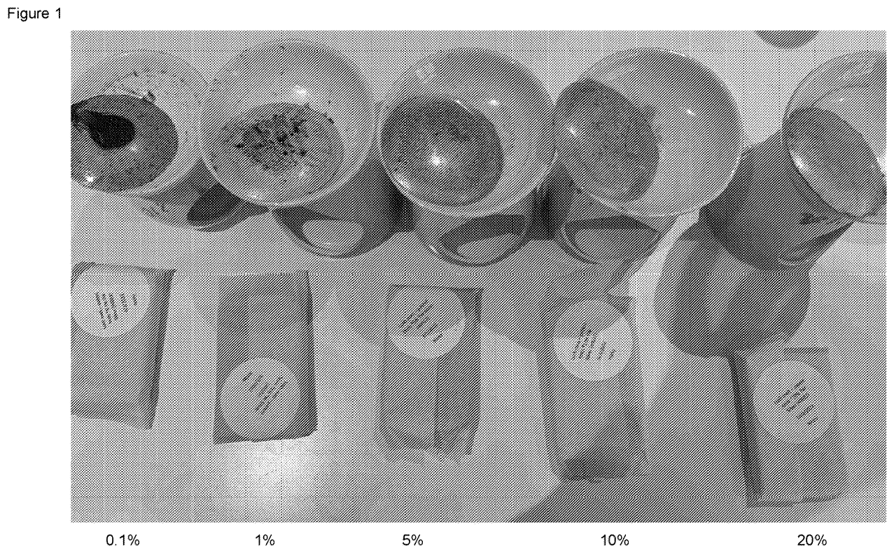

The results are shown in FIG. 1.

### Trial 2

0.1% no effect
1% limited coating of the bottom of the solidified product
5% very good
10% very good
20% removal but brittle texture when set

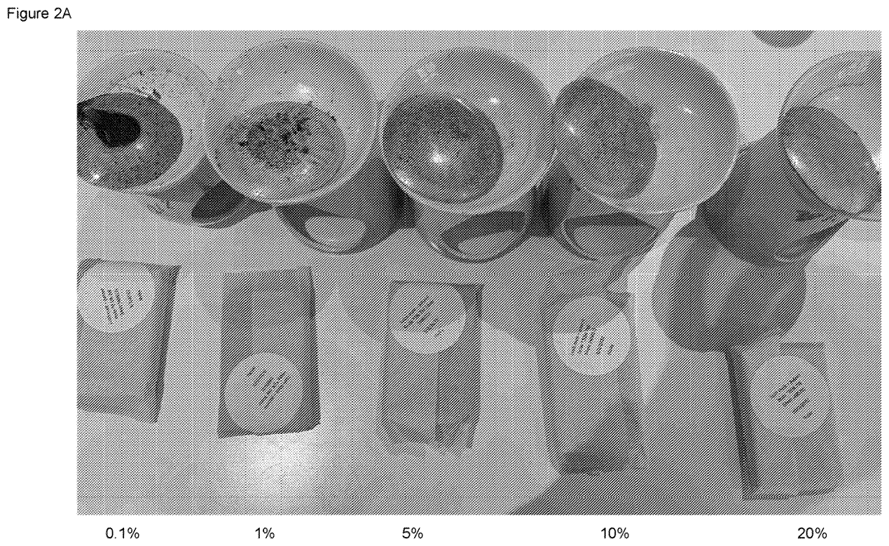

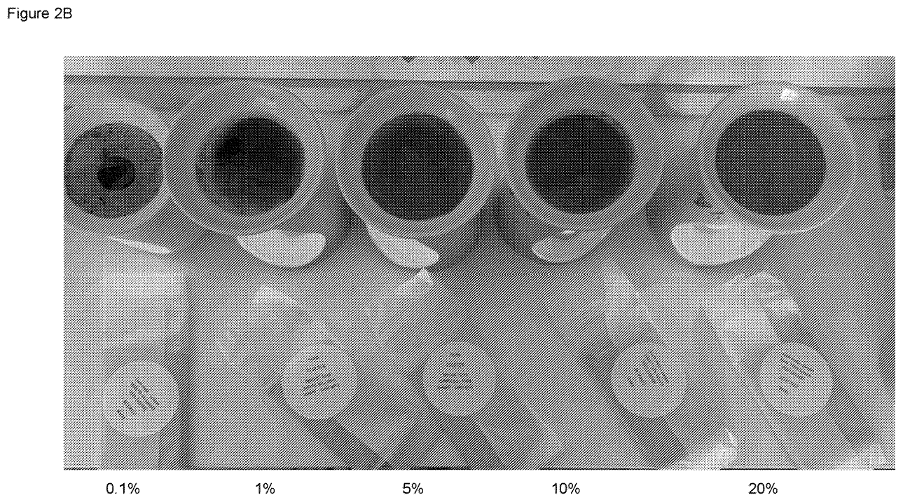

The results are shown in FIGS. 2A and 2B.

### Trial 3

0.1% not enough
1% not enough
5% very good
10% very good
20% made the texture breakable

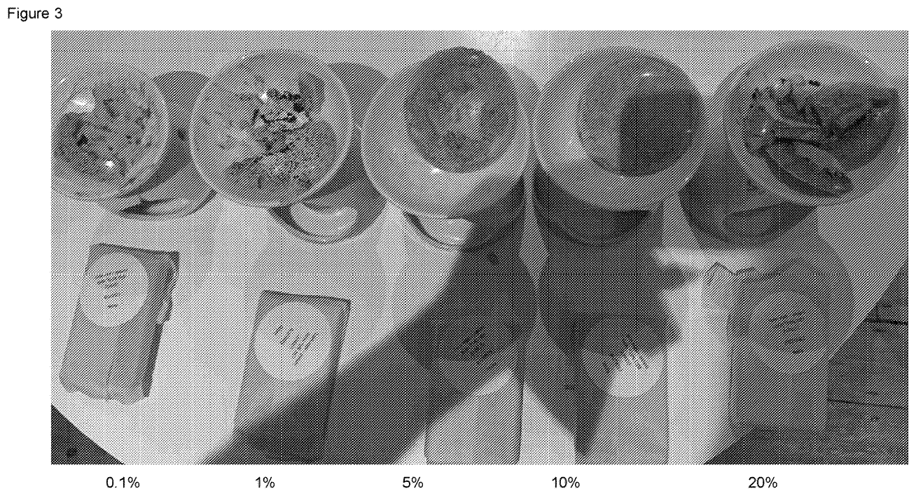

The results are shown in FIG. 3.

### Trial 4

0.1% not enough
1% enough
5% optimum
10% acceptable
20% caused reduction in the setting of the composition

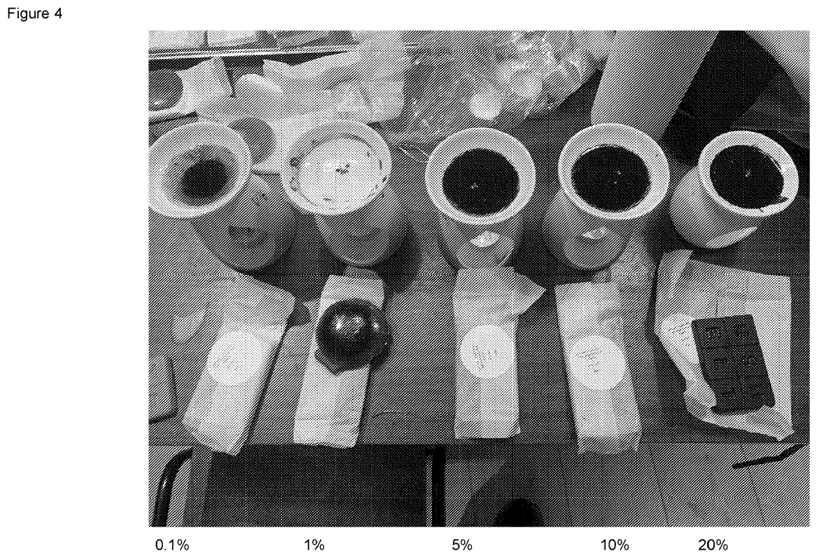

The results are shown in FIG. 4.

### Trial 5

0.10% not enough
1% enough
5% optimal
10% great
20% acceptable but some separation/precipitation at the bottom of the set wax pool and residue floating when burning

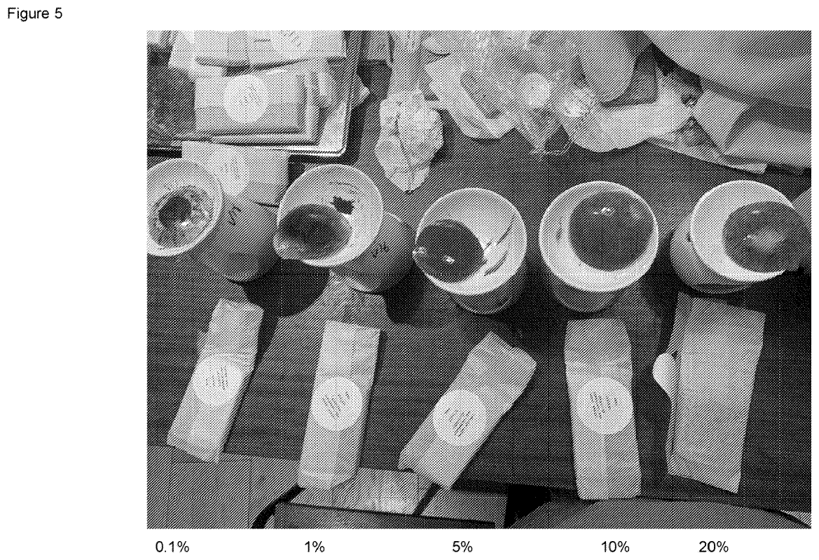

The results are shown in FIG. 5.

### Trial 6

0.1% not enough
1% acceptable
5% perfect
10% good
20% also good—although some residue floating to the surface whilst burning

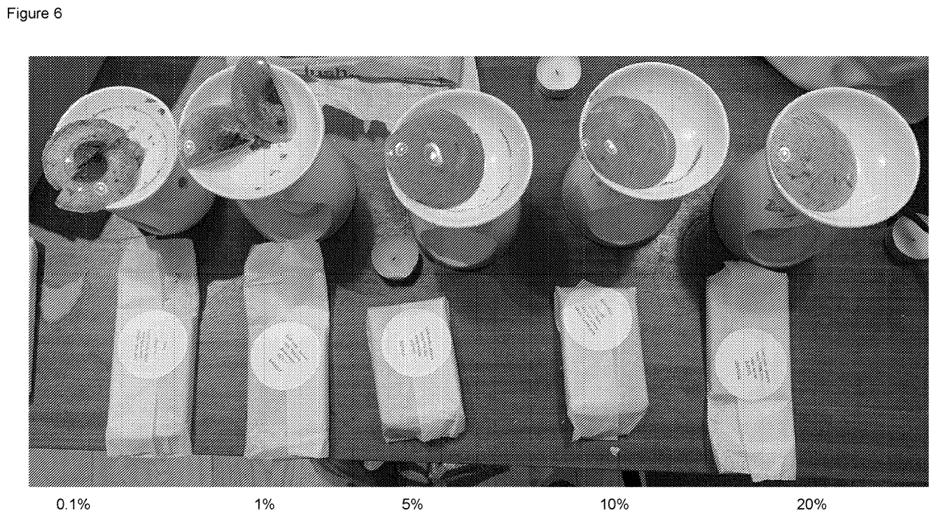

The results are shown in FIG. 6.

As will be appreciated, the trials showed that the present combination of emulsifiers and inert powder at the cited amounts provided a composition that would melt and when cooled with solidify and allow for removal from the heating device.

Furthermore, each of the products may be placed in warm bathing water and found to readily disperse to provide bathing water containing emollient vegetable butter and pleasing colour.

Various modifications and variations of the present invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry, biology or related fields are intended to be within the scope of the following claims.

1. A solid cosmetic composition comprising:
(a) vegetable butter;
(b) a first emulsifier having a hydrophilic-lipophilic balance (HLB) value of from 2 to 9;
(c) a second emulsifier having an HLB value of from 10 to 20;
(d) one or more inert powders in an amount of from 0.5 to 25 wt. % based on the solid cosmetic composition;
(e) one or more colours; and
(f) one or more fragrances.

2. A solid cosmetic composition according to claim 1 wherein vegetable butter is present in an amount of from 30 to 80 wt. % based on the solid cosmetic composition.

3. A solid cosmetic composition according to claim 1 wherein vegetable butter is present in an amount of from 40 to 70 wt. % based on the solid cosmetic composition.

4. A solid cosmetic composition according to claim 1, wherein the vegetable butter is selected from cocoa butter, illipe butter, murumuru butter, kokum butter, aloe butter, avocado butter, cupuacu butter, macadamia nut butter, mango butter, olive butter, shea butter, coconut butter, pumpkin seed butter, peanut butter, almond butter, coffee bean butter, refined butter, hemp seed butter, mochacchino butter, pistachio nut butter, shealoe butter and mixtures thereof.

5. A solid cosmetic composition according to claim 1, wherein the first emulsifier has an HLB value of from 4 to 9.

6. A solid cosmetic composition according to claim 1, wherein the first emulsifier is an emulsifier having an HLB value of from 2 to 9 selected from mono and diglycerydes of fatty acid including glyceryl stearate and glyceryl oleate; fatty acid esters of C12-C22 fatty alcohols including fatty acid esters of cetyl alcohol and fatty acid esters of stearoyl alcohol, mixtures of fatty acid esters of cetyl alcohol and fatty acid esters of stearoyl alcohol, mixtures of fatty acid esters of cetyl alcohol and fatty acid esters of stearoyl alcohol wherein the fatty acids are derived from olive oil (such as cetearly olivate), fatty acid esters of sorbitol including sorbitan oleate, fatty acid esters of sorbitol wherein the fatty acids are derived from olive oil (such as sorbitan olivate or cetearyl olivate), and mixtures thereof.

7. A solid cosmetic composition according to claim 1, wherein the first emulsifier is present in an amount of from 5 to 30 wt. % based on the solid cosmetic composition.

8. A solid cosmetic composition according to claim 1, wherein the first emulsifier is present in an amount of from 10 to 20 wt. % based on the solid cosmetic composition.

9. A solid cosmetic composition according to claim 1, wherein the second emulsifier has an HLB value of from 10 to 13.

10. A solid cosmetic composition according to claim 1, wherein the second emulsifier is an emulsifier having an HLB value of from 10 to 20 selected from fatty acid esters of polyethylene glycol, such as fatty acid esters of polyethylene glycol wherein the fatty acids are derived from coconut oil, fatty acid esters of polyglycerol including fatty acid esters of polyglycerol and oleic acid, and mixtures thereof.

11. A solid cosmetic composition according to claim 1, wherein the second emulsifier is present in an amount of from 5 to 30 wt. % based on the solid cosmetic composition.

12. A solid cosmetic composition according to claim 1, wherein the second emulsifier is present in an amount of from 10 to 20 wt. % based on the solid cosmetic composition.

13. A solid cosmetic composition according to claim 1, wherein the one or more inert powders is selected from silicates, such as phyllosilicates, such as mica, such as fluorphlogopite, and such as synthetic fluorphlogopite; titanium dioxide; tin oxide; zinc oxide; silicas, such as glasses, such as borosilicates and such as calcium sodium borosilicate; zeolites including zeolite containing polysulfides, such as sodium aluminosilicate and such as Al₆Na₆O₂₄S₈Si₆; hydrated silica; metal carbonates such as sodium carbonate, calcium carbonate, sodium bicarbonate and mixtures thereof, silicates, such as layered silicates and such as kaolin; and mixtures thereof.

14. A solid cosmetic composition according to claim 1, wherein the one or more inert powders is selected from phyllosilicates, such as mica, such as fluorphlogopite, and such as synthetic fluorphlogopite; titanium dioxide; tin oxide; zinc oxide; borosilicates such as calcium sodium borosilicate; zeolites such as sodium aluminosilicate and such as Al₆Na₆O₂₄S₈Si₆; hydrated silica; metal carbonates such as sodium carbonate, calcium carbonate, sodium bicarbonate and mixtures thereof; layered silicates such as kaolin; and mixtures thereof.

15. A solid cosmetic composition according to claim 1, wherein the one or more inert powders is selected from mica, such as fluorphlogopite, and such as synthetic fluorphlogopite; titanium dioxide; tin oxide; zinc oxide; calcium sodium borosilicate; sodium aluminosilicate such as Al₆Na₆O₂₄S₈Si₆; hydrated silica; sodium carbonate; calcium carbonate; sodium bicarbonate; kaolin; and mixtures thereof.

16. A solid cosmetic composition according to claim 1, wherein the one or more inert powders is selected from synthetic fluorphlogopite; titanium dioxide; zinc oxide; sodium aluminosilicate; hydrated silica; and mixtures thereof.

17. A solid cosmetic composition according to claim 1, wherein the one or more inert powders comprises at least hydrated silica.

18. A solid cosmetic composition according to claim 1, wherein the one or more inert powders consists of hydrated silica.

19. A solid cosmetic composition according to claim 1, wherein the one or more inert powders are present in an amount of from 1 to 25 wt. % based on the solid cosmetic composition.

20. A solid cosmetic composition according to claim 1, wherein the one or more inert powders are present in an amount of from 5 to 20 wt. % based on the solid cosmetic composition.

21. A solid cosmetic composition according to claim 1, wherein the colour is selected from water soluble colour, oil soluble colour, and mixtures thereof.

22. A solid cosmetic composition according to claim 1, wherein the colour is at least one water soluble colour.

23. A solid cosmetic composition according to claim 1, wherein the composition contains colour in an amount of from 0.001 to 1 wt. % based on the solid cosmetic composition.

24. A solid cosmetic composition according to claim 1, wherein the composition contains colour in an amount of from 0.01 to 0.5 wt. % based on the solid cosmetic composition.

25. A solid cosmetic composition comprising
(a) vegetable butter;
(b) a first emulsifier having hydrophilic-lipophilic balance (HLB) value of from 2 to 9;
(c) a second emulsifier having an HLB value of from 10 to 20;
(d) hydrated silica;
(e) one or more colours; and
(f) one or more fragrances.

26. A process for the production of a solid cosmetic composition as defined in claim 1, the process comprising the steps of:
(i) preparing a first composition comprising
(a) vegetable butter; and
(b) a first emulsifier having an HLB value of from 2 to 9;
(ii) preparing a second composition comprising
(c) a second emulsifier having an HLB value of from 10 to 20;
(d) one or more inert powders;
(e) one or more colours;
(f) one or more fragrances;
(iii) combining the first composition with the second composition, to form the solid cosmetic composition; wherein the one or more inert powders are present in an amount of from 0.5 to 25 wt. % based on the solid cosmetic composition.

27. A product obtained or obtainable by the process of claim 26.
